(19) United States
(12) Patent Application Publication
(10) Pub. No.: US 2026/0207175 A1
(43) Pub. Date: Jul. 23, 2026

(54) ULTRASOUND IMAGING APPARATUS FOR DISPLAYING ULTRASOUND VIDEO AND METHOD THEREOF

(71) Applicant: Samsung Medison Co., Ltd., Hongcheon-gun (KR)

(72) Inventors: Jaehong Lee, Seoul (KR); Taewoo Kwon, Seoul (KR)

(73) Assignee: SAMSUNG MEDISON CO., LTD., Hongcheon-gun (KR)

(21) Appl. No.: 19/186,368

(22) Filed: Apr. 22, 2025

(30) Foreign Application Priority Data
Jan. 21, 2025 (KR) 10-2025-0008849

(51) Int. Cl. A61B 8/00; G06T 7/00; G06T 7/215

(52) U.S. Cl. CPC A61B 8/5207; A61B 8/461; A61B 8/469; A61B 8/54; G06T 7/0012; G06T 7/215; A61B 8/481; A61B 8/485; A61B 8/486; A61B 8/488; G06T 2207/10016; G06T 2207/10024; G06T 2207/10132; G06T 2207/20081

(57) ABSTRACT

Provided are an ultrasound imaging apparatus for displaying an ultrasound video, and a method thereof. In detail, provided are an ultrasound imaging apparatus and a method thereof, the ultrasound imaging apparatus configured to adjust a reproduction speed of a section of interest in an ultrasound video, change a frame rate of the ultrasound video for the section of interest from a first frame rate to a second frame rate, based on the adjusted reproduction speed, input a plurality of frame images included in the section of interest to an artificial intelligence model, generate at least one interpolation frame between the plurality of frame images by using the artificial intelligence model, generate an interpolated ultrasound video based on the plurality of frame images and the at least one interpolation frame, and display the interpolated ultrasound video through a display.

### CROSS-REFERENCE TO RELATED APPLICATION

This application is based on and claims priority under 35 U.S.C. § 119 to Korean Patent Application No. 10-2025-0008849, filed on Jan. 21, 2025, in the Korean Intellectual Property Office, the disclosure of which is incorporated by reference herein in its entirety.

### BACKGROUND

### 1. Field

The disclosure relates to an ultrasound imaging apparatus for displaying an ultrasound video, and a method thereof. More particularly, the disclosure relates to a technique for providing an enhanced-quality ultrasound video by estimating a movement of an object in an ultrasound video by using a frame interpolation technique based on artificial intelligence such as deep learning.

### 2. Description of the Related Art

Recently, in the medical field, various medical imaging apparatuses are widely used for imaging and obtaining information about living tissues of a human body for early diagnosis or surgery of various diseases. Representative examples of such medical imaging apparatuses may include ultrasound imaging apparatuses, computed tomography (CT) apparatuses, and magnetic resonance imaging (MRI) apparatuses.

An ultrasound imaging apparatus emits an ultrasound signal generated from a transducer of a probe toward an object, and receives information about the signal reflected from the object, so as to non-invasively obtain at least one video with respect to an internal region of the object (e.g., soft tissue or blood flow). An ultrasound imaging apparatus may be used for medical purposes such as observation of an interior of an object, detection of foreign objects, or assessment of injury. Such ultrasound imaging apparatuses offer advantages of higher stability, real-time video display, and safety without radiation exposure compared to imaging devices using X-rays, and thus are widely used alongside other imaging apparatuses.

An ultrasound imaging apparatus may display an ultrasound video. The ultrasound video may be a video in which a plurality of frames are displayed sequentially. When displaying an ultrasound video by using a related-art ultrasound imaging apparatus, intermediate frames are generated between a plurality of consecutive frames by using an optical flow-based frame interpolation technique. An optical flow-based frame interpolation technique may calculate an optical flow between two consecutive frames, warp the two frames with respect to each other, and blend the warped frames with each other to generate an intermediate frame.

When using an optical flow-based frame interpolation technique, it is difficult to display an object that is visible in one frame but not in another. Furthermore, it is difficult to calculate an optical flow of an object with a complex or fast movement by using an optical flow-based frame interpolation technique. Accordingly, when displaying a rapidly changing ultrasound video by using an optical flow-based frame interpolation technique, a degradation of the image quality may occur.

### SUMMARY

Additional aspects will be set forth in part in the description which follows and, in part, will be apparent from the description, or may be learned by practice of the presented embodiments of the disclosure.

According to an embodiment, an ultrasound imaging apparatus for displaying an ultrasound video includes an ultrasound transceiver module configured to obtain volume data for displaying the ultrasound video, a display configured to display the ultrasound video, a memory storing at least one instruction, and at least one processor electrically connected to the ultrasound transceiver module, the display, and the memory, wherein the at least one processor is configured to execute the at least one instruction to cause the ultrasound imaging apparatus to adjust a reproduction speed of a section of interest in the ultrasound video, change a frame rate of the ultrasound video for the section of interest from a first frame rate to a second frame rate, based on the adjusted reproduction speed, input a plurality of frame images included in the section of interest to an artificial intelligence model, generate at least one interpolation frame between the plurality of frame images by using the artificial intelligence model, generate an interpolated ultrasound video based on the plurality of frame images and the at least one interpolation frame, and display the interpolated ultrasound video through the display.

According to an embodiment, a method, performed by an ultrasound imaging apparatus, of displaying an ultrasound video includes adjusting a reproduction speed of a section of interest in the ultrasound video, changing a frame rate of the ultrasound video for the section of interest from a first frame rate to a second frame rate, based on the adjusted reproduction speed, inputting a plurality of frame images included in the section of interest to an artificial intelligence model, generating at least one interpolation frame between the plurality of frame images by using the artificial intelligence model, generating an interpolated ultrasound video based on the plurality of frame images and the at least one interpolation frame, and displaying the interpolated ultrasound video.

### BRIEF DESCRIPTION OF THE DRAWINGS

The disclosure may be readily understood with a combination of the following detailed descriptions and the accompanying drawings, wherein reference numbers refer to structural elements.

The above and other aspects, features, and advantages of certain embodiments of the disclosure will be more apparent from the following description taken in conjunction with the accompanying drawings, in which:

FIGS. 1A and 1B are block diagrams illustrating configurations of an ultrasound imaging system according to an embodiment;

FIGS. 2A, 2B, 2C, and 2D are diagrams each illustrating an ultrasound imaging apparatus according to an embodiment;

FIG. 3 is a block diagram illustrating a processor of an ultrasound imaging apparatus according to an embodiment;

FIG. 4 is a flowchart of a method, performed by an ultrasound imaging apparatus, of displaying an ultrasound video, according to an embodiment;

FIG. 5 is a flowchart of a method, performed by an ultrasound imaging apparatus, of processing an ultrasound video, according to an embodiment;

FIG. 6 is a diagram illustrating an original ultrasound video displayed by an ultrasound imaging apparatus, according to an embodiment;

FIG. 7 is a diagram illustrating an ultrasound imaging apparatus, according to the disclosure, displaying an ultrasound video that is interpolated according to a comparative example;

FIG. 8 is a diagram illustrating an ultrasound imaging apparatus, according to the disclosure, displaying an ultrasound video that is interpolated according to an embodiment;

FIG. 9 is a diagram illustrating an ultrasound imaging apparatus interpolating a portion of an ultrasound video, according to an embodiment;

FIG. 10 is a flowchart of a method, performed by an ultrasound imaging apparatus, of extracting a section of interest, according to an embodiment;

FIG. 11 is a flowchart of a method, performed by an ultrasound imaging apparatus, of changing a frame rate, according to an embodiment;

FIG. 12 is a flowchart of a method, performed by an ultrasound imaging apparatus, of generating an interpolation frame, according to an embodiment;

FIG. 13 is a diagram illustrating an ultrasound imaging apparatus setting a region of interest, according to an embodiment;

FIG. 14 is a diagram illustrating an ultrasound imaging apparatus displaying an interpolated M-mode video, according to an embodiment;

FIG. 15 is a diagram illustrating an ultrasound imaging apparatus displaying a color Doppler video, according to an embodiment;

FIG. 16 is a flowchart illustrating an ultrasound imaging apparatus displaying a result of combining an elasticity video with a B-mode video, according to an embodiment; and

FIG. 17 is a flowchart illustrating an ultrasound imaging apparatus interpolating a contrast-enhanced ultrasound (CEUS) video, according to an embodiment.

### DETAILED DESCRIPTION

To clarify the scope of the claims of the disclosure and to enable those of skill in the art to which embodiments belong to practice the embodiments, the principles of the embodiments will be described and disclosed. The embodiments may be implemented in various forms.

Like reference numerals denote like elements throughout the specification. The specification does not describe all elements of embodiments, and general content in the art to which the disclosure pertains or identical content between the embodiments will be omitted. A “module” or “unit” used herein may be implemented with software, hardware, firmware, or a combination thereof, and depending on embodiments, a plurality of “modules” or “units” may be implemented as one element, or one “module” or “unit” may include a plurality of elements.

A singular form of a noun corresponding to an item may include one or more of the things, unless the relevant context clearly indicates otherwise.

As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases.

As used herein, the term “and/or” includes any one or a combination of a plurality of related recited elements.

As used herein, such terms as “1st” and “2nd,” or “first” and “second” may be used to simply distinguish a corresponding component from another, and does not limit the components in any other respect (e.g., importance or order).

In addition, as used herein, such terms as “front surface”, “rear surface”, “upper surface”, “lower surface”, “side surface”, “left”, “right”, “upper”, or “lower” are defined with respect to the drawings, and the shape and position of each component are not limited by these terms.

As used herein, such terms as “comprises,” “includes,” or “has” specify the presence of stated features, numbers, stages, operations, components, parts, or a combination thereof, but do not preclude the presence or addition of one or more other features, numbers, stages, operations, components, parts, or a combination thereof.

When an element is referred to as being “connected to,” “coupled to,” “supported by,” or “in contact with” another element, it means that the element is directly connected to, coupled to, supported by, or in contact with the other element, or that the element is indirectly connected to, coupled to, supported by, or in contact with the other element via a third element.

When an element is referred to as being “on” another element, it means that the element is in contact with the other element, or that still another element is present between the element and the other element.

Hereinafter, an ultrasound imaging apparatus according to various embodiments will be described in detail with reference to the accompanying drawings. In the description with reference to the accompanying drawings, identical or corresponding components may be given similar reference numerals, and redundant descriptions thereof may be omitted.

In the disclosure, the term “video” may include medical videos obtained by a medical imaging apparatus such as a magnetic resonance imaging (MRI) apparatus, a computed tomography (CT) apparatus, an ultrasound imaging apparatus, or an X-ray imaging apparatus.

In the present disclosure, the term “object” refers to a target to be imaged, and may include a human, an animal, or part thereof. For example, the term “object” may include part of a human body (e.g., an organ), a phantom, and the like.

In the disclosure, the term “ultrasound video” refers to a video with respect to an object generated or processed based on an ultrasound signal transmitted to and reflected from the object.

Hereinafter, embodiments will be described in detail with reference to the drawings.

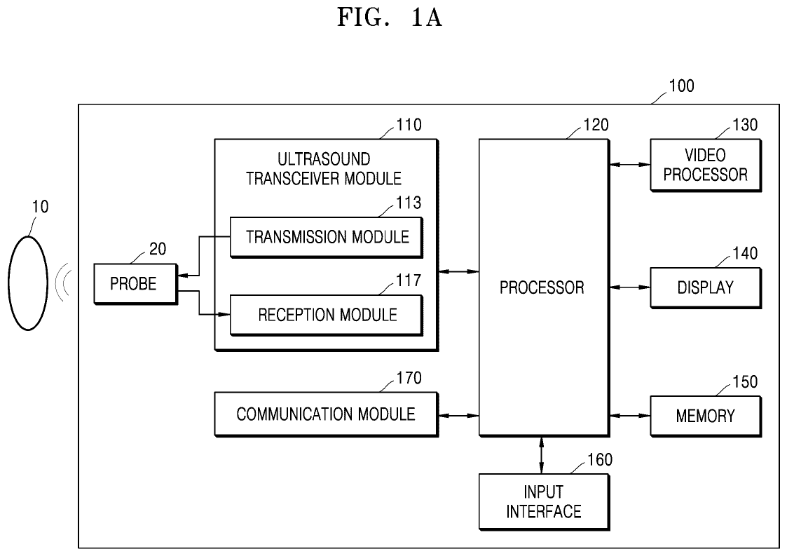

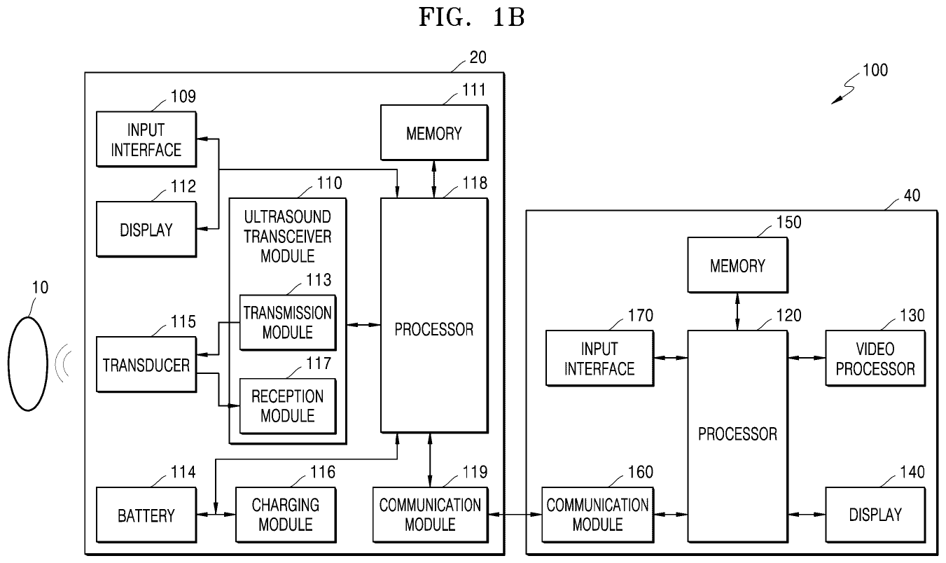

FIGS. 1A and 1B are block diagrams illustrating configurations of an ultrasound imaging system according to an embodiment.

Referring to FIGS. 1A and 1B, an ultrasound imaging system 100 may include a probe 20 and an ultrasound imaging apparatus 40.

The ultrasound imaging apparatus 40 may be implemented not only as a cart type but also as a portable type. Examples of portable ultrasound imaging apparatuses may include a smart phone, a laptop computer, a personal digital assistant (PDA), or a tablet personal computer (PC) including a probe and an application, but are not limited thereto. The ultrasound imaging apparatus 40 may also be implemented as a probe-integrated type.

The probe 20 may include a wired probe configured to connect to and communicate with the ultrasound imaging apparatus 40 by wire, a wireless probe configured to connect to and communicate with the ultrasound imaging apparatus 40 wirelessly, and/or a hybrid probe configured to connect to and communicate with the ultrasound imaging apparatus 40 by wire or wirelessly.

According to various embodiments, as illustrated in FIG. 1A, the ultrasound imaging apparatus 40 may include an ultrasound transceiver module 110, or as illustrated in FIG. 1B, the probe 20 may include the ultrasound transceiver module 110. According to various embodiments, it is also possible that both the ultrasound imaging apparatus 40 and the probe 20 include the ultrasound transceiver module 110.

According to various embodiments, the probe 20 may further include at least one of a video processor 130, a display 140, or an input interface 170, or a combination thereof. In the disclosure, the description of the ultrasound transceiver module 110, the video processor 130, the display 140, or the input interface 170 included in the ultrasound imaging apparatus 40 may also be applied to the ultrasound transceiver module 110, the video processor 130, the display 140, or the input interface 170 included in the probe 20.

FIG. 1A is a block diagram illustrating a configuration of the ultrasound imaging system 100 in a case in which the probe 20 is a wired probe or a hybrid probe.

The probe 20 may include a plurality of transducers. The plurality of transducers may be provided in a certain arrangement to form a transducer array. The transducer array may correspond to a one-dimensional (1D) array or a two-dimensional (2D) array. The plurality of transducers may transmit an ultrasound signal to an object 10 according to a transmission signal applied from a transmission module 113. The plurality of transducers may receive an ultrasound signal (echo signal) reflected from the object 10 to form a reception signal. In addition, the probe 20 may be implemented integrally with the ultrasound imaging apparatus 40 or as a separate unit connected to the ultrasound imaging apparatus 40 by wire. In addition, the ultrasound imaging apparatus 40 may be connected to one or more probes 20 according to an implementation.

In a case in which the probe 20 is a wired probe or a hybrid probe, the probe 20 may include a cable and a connector connectable to a connector of the ultrasound imaging apparatus 40.

The probe 20 according to an embodiment may be implemented as a 2D probe. In a case in which the probe 20 is implemented as a 2D probe, the plurality of transducers included in the probe 20 may be arranged two-dimensionally to form a 2D transducer array.

For example, the 2D transducer array may include a plurality of sub-arrays, each including a plurality of transducers arranged in a first direction, with the sub-arrays arranged in a second direction different from the first direction.

In addition, in a case in which the probe 20 according to an embodiment is implemented as a 2D probe, the ultrasound transceiver module 110 may include at least one of an analog beamformer or a digital beamformer. In addition, according to an embodiment, the 2D probe may include at least one of an analog beamformer or a digital beamformer, or a combination thereof, according to an implementation.

A processor 120 controls the transmission module 113 to form a transmission signal to be applied to each transducer 115, considering the positions and focal points of the plurality of transducers included in the probe 20.

The processor 120 may control a reception module 117 to perform analog-to-digital conversion on reception signals received from the probe 20, and generate ultrasound data by summing the reception signals that have undergone the analog-to-digital conversion, considering the positions and focal points of the plurality of transducers.

In a case in which the probe 20 is implemented as a 2D probe, the processor 120 may calculate a time delay value for digital beamforming for each of a plurality of sub-arrays included in a 2D transducer array, on a per-sub-array basis. In addition, the processor 120 may calculate a time delay value for analog beamforming for each of transducers included in any one of the plurality of sub-arrays. The processor 120 may control an analog beamformer and a digital beamformer to form transmission signals to be applied to the plurality of transducers, respectively, according to the time delay values for analog beamforming and the time delay values for digital beamforming. In addition, the processor 120 may control the analog beamformer to sum signals received from the plurality of transducers for each sub-array, according to the time delay values for analog beamforming. In addition, the processor 120 may control the ultrasound transceiver module 110 to perform analog-to-digital conversion on a result of summing the signals for each sub-array. In addition, the processor 120 may control the digital beamformer to generate ultrasound data by summing the signals that have undergone the analog-to-digital conversion, according to the time delay values for digital beamforming.

The video processor 130 generates or processes an ultrasound video by using the generated ultrasound data.

The display 140 may display the generated ultrasound video and various pieces of information processed by the ultrasound imaging apparatus 40 or the probe 20. The probe 20 or the ultrasound imaging apparatus 40 may include one or more displays 140 according to an implementation. In addition, the display 140 may include a touch panel or a touch screen. In addition, the display 140 may include a flexible display.

The processor 120 may control the overall operation of the ultrasound imaging apparatus 40, and may control operations of the components of the ultrasound imaging apparatus 40. The processor 120 may execute programs or instructions stored in a memory 150 to perform or control various operations or functions of the ultrasound imaging apparatus 40. In addition, the processor 120 may receive a control signal from the input interface 170 or an external device, to control an operation of the ultrasound imaging apparatus 40.

The ultrasound imaging apparatus 40 may include a communication module 160, and may connect to and communicate with external devices (e.g., the probe 20, a server, a medical device, or a portable device (e.g., a smart phone, a tablet PC, or a wearable device)) through the communication module 160.

The communication module 160 may include one or more components that enable communication with an external device. The communication module 160 may include, for example, at least one of a short-range communication module, a wired communication module, or a wireless communication module.

The communication module 160 may receive a control signal or data from an external device. The processor 120 may control an operation of the ultrasound imaging apparatus 40 according to a control signal received through the communication module 160. In addition, the processor 120 may transmit a control signal to an external device through the communication module 160 to control the external device according to the transmitted control signal. The external device may operate according to a control signal received from the ultrasound imaging apparatus 40 or may process data received from the ultrasound imaging apparatus 40.

A program or an application associated with the ultrasound imaging apparatus 40 may be installed on the external device. The program or application installed on the external device may control the ultrasound imaging apparatus 40 or may operate according to a control signal or data received from the ultrasound imaging apparatus 40.

The external device may receive or download a program or an application associated with the ultrasound imaging apparatus 40 from the ultrasound imaging apparatus 40, the probe 20, or a server, and install and execute the program or application on the external device. The ultrasound imaging apparatus 40, the probe 20, or the server providing the program or application may include a recording medium storing instructions, commands, installation files, executable files, data, or the like associated with the program or application. The external device may also be sold with the program or application pre-installed.

The memory 150 may store various pieces of data or programs for driving and controlling the ultrasound imaging apparatus 40, input/output ultrasound data, ultrasound videos, and the like.

The input interface 170 may receive a user input for controlling the ultrasound imaging apparatus 40. For example, the user input may include, but is not limited to, an input of manipulating a button, a keypad, a mouse, a trackball, a jog switch, a knob, or the like, an input of touching a touch pad or a touch screen, a voice input, a motion input, a biometric information input (e.g., iris recognition or fingerprint recognition), and the like.

FIG. 1B is a control block diagram of the ultrasound imaging system 100 in a case in which the probe 20 is a wireless probe or a hybrid probe.

According to various embodiments, the ultrasound imaging apparatus 40 illustrated in FIG. 1B may be replaced with the ultrasound imaging apparatus 40 described above with reference to FIG. 1A.

According to various embodiments, the probe 20 illustrated in FIG. 1A may be replaced with the probe 20 described above with reference to FIG. 1B.

The probe 20 may include a display 112, the transmission module 113, a battery 114, the transducer 115, a charging module 116, the reception module 117, an input interface 109, a processor 118, and a communication module 119. FIG. 1B illustrates that the probe 20 includes both the transmission module 113 and the reception module 117, however, according to an implementation, the probe 20 may include only some of the transmission module 113 and the reception module 117, and some of the transmission module 113 and the reception module 117 may be included in the ultrasound imaging apparatus 40. In addition, according to an embodiment, the probe 20 may further include the video processor 130.

The transducer 115 may include a plurality of transducers. The plurality of transducers may be provided in a certain arrangement to form a transducer array. The transducer array may correspond to a 1D array or a 2D array. The plurality of transducers may transmit an ultrasound signal to the object 10 according to a transmission signal applied from a transmission module 113. In addition, the plurality of transducers may receive an ultrasound signal reflected from the object 10 to form or generate an electrical reception signal.

The charging module 116 may charge the battery 114. The charging module 116 may receive power from an external source. According to an embodiment, the charging module 116 may receive power wirelessly. In addition, according to an embodiment, the charging module 116 may also receive power by wire. The charging module 116 may deliver the received power to the battery 114.

The processor 118 controls the transmission module 113 to generate or form a transmission signal to be applied to each of the plurality of transducers, considering the positions and focal points of the plurality of transducers.

The processor 118 controls the reception module 117 to perform analog-to-digital conversion on a reception signal received from the transducer 115, and generate ultrasound data by summing reception signals that have undergone the analog-to-digital conversion, considering the positions and focal points of the plurality of transducers. According to an embodiment, in a case in which the probe 20 includes the video processor 130, an ultrasound video may be generated by using the generated ultrasound data.

In a case in which the probe 20 is implemented as a 2D probe, the processor 118 may calculate a time delay value for digital beamforming for each of a plurality of sub-arrays included in a 2D transducer array, on a per-sub-array basis. In addition, the processor 118 may calculate a time delay value for analog beamforming for each of transducers included in any one of the plurality of sub-arrays. The processor 118 may control an analog beamformer and a digital beamformer to form transmission signals to be applied to the plurality of transducers, respectively, according to the time delay values for analog beamforming and the time delay values for digital beamforming. In addition, the processor 118 may control the analog beamformer to sum signals received from the plurality of transducers for each sub-array, according to the time delay values for analog beamforming. In addition, the processor 118 may control the ultrasound transceiver module 110 to perform analog-to-digital conversion on a result of summing the signals for each sub-array. In addition, the processor 118 may control the digital beamformer to generate ultrasound data by summing the signals that have undergone the analog-to-digital conversion, according to the time delay values for digital beamforming.

The processor 118 may control the overall operation of the probe 20, and may control operations of the components of the probe 20. The processor 118 may execute programs or instructions stored in a memory 111 to perform or control various operations or functions of the probe 20. In addition, the processor 118 may receive a control signal from the input interface 109 of the probe 20 or from an external device (e.g., the ultrasound imaging apparatus 40) to control an operation of the probe 20. In addition, the processor 118 may receive a control signal from the input interface 109 or an external device to control an operation of the probe 20. The input interface 109 may receive a user input for controlling the probe 20. For example, the user input may include, but is not limited to, an input of manipulating a button, a keypad, a mouse, a trackball, a jog switch, a knob, or the like, an input of touching a touch pad or a touch screen, a voice input, a motion input, a biometric information input (e.g., iris recognition or fingerprint recognition), and the like.

The display 112 may display an ultrasound video generated by the probe 20, an ultrasound video generated by processing ultrasound data generated by the probe 20, an ultrasound video received from the ultrasound imaging apparatus 40, various pieces of information processed by the ultrasound imaging system 100, or the like. In addition, the display 112 may further display status information about the probe 20. The status information about the probe 20 may include at least one of device information about the probe 20, battery status information about the probe 20, frequency band information about the probe 20, output information about the probe 20, information about the presence or absence of an abnormality of the probe 20, setting information about the probe 20, or temperature information about the probe 20.

The probe 20 may include one or more displays 112 according to an implementation. In addition, the display 112 may include a touch panel or a touch screen. In addition, the display 112 may include a flexible display.

The communication module 119 may wirelessly transmit generated ultrasound data or ultrasound video to the ultrasound imaging apparatus 40 via a wireless network. In addition, the communication module 119 may receive a control signal and data from the ultrasound imaging apparatus 40.

The ultrasound imaging apparatus 40 may receive ultrasound data or an ultrasound video from the probe 20.

In an embodiment, in a case in which the probe 20 includes the video processor 130 capable of generating an ultrasound video by using ultrasound data, the probe 20 may transmit ultrasound data or an ultrasound video generated by the video processor 130 to the ultrasound imaging apparatus 40.

In an embodiment, in a case in which the probe 20 does not include the video processor 130 capable of generating an ultrasound video by using ultrasound data, the probe 20 may transmit ultrasound data to the ultrasound imaging apparatus 40. Ultrasound data may include raw ultrasound data, and an ultrasound video may refer to ultrasound video data.

The ultrasound imaging apparatus 40 may include the processor 120, the video processor 130, the display 140, the memory 150, the communication module 160, and the input interface 170.

The video processor 130 generates or processes an ultrasound video by using ultrasound data received from the probe 20.

The display 140 may display an ultrasound video received from the probe 20, an ultrasound video generated by processing ultrasound data received from the probe 20, various pieces of information processed by the ultrasound imaging system 100, or the like. The ultrasound imaging apparatus 40 may include one or more displays 140 according to an implementation. In addition, the display 140 may include a touch panel or a touch screen. In addition, the display 140 may include a flexible display.

The processor 120 may control the overall operation of the ultrasound imaging apparatus 40, and may control operations of the components of the ultrasound imaging apparatus 40. The processor 120 may execute a program or an application stored in the memory 150 to perform or control various operations or functions of the ultrasound imaging apparatus 40. In addition, the processor 120 may receive a control signal from the input interface 170 or an external device, to control an operation of the ultrasound imaging apparatus 40.

The ultrasound imaging apparatus 40 may include the communication module 160, and may connect to and communicate with external devices (e.g., the probe 20, a server, a medical device, or a portable device (e.g., a smart phone, a tablet PC, or a wearable device)) through the communication module 160.

The communication module 160 may include one or more components that enable communication with an external device. The communication module 160 may include, for example, at least one of a short-range communication module, a wired communication module, or a wireless communication module.

The communication module 160 of the ultrasound imaging apparatus 40 and the communication module 119 of the probe 20 may perform communication by using a network and may perform communication by using a short-range wireless communication method. For example, the communication module 160 of the ultrasound imaging apparatus 40 and the communication module 119 of the probe 20 may perform communication by using any one of wireless data communication methods, including wireless local area network (LAN), Wi-Fi, Bluetooth, Zigbee, Wi-Fi Direct (WFD), Infrared Data Association (IrDA), Bluetooth Low Energy (BLE), near-field communication (NFC), Wireless Broadband Internet (WiBro), Worldwide Interoperability for Microwave Access (WiMAX), Shared Wireless Access Protocol (SWAP), Wireless Gigabit Alliance (WiGig), radio-frequency (RF) communication, or 60-GHz millimeter wave (mmWave) short-range communication.

To this end, the communication module 160 of the ultrasound imaging apparatus 40 and the communication module 119 of the probe 20 may include at least one of a wireless LAN communication module, a Wi-Fi communication module, a Bluetooth communication module, a Zigbee communication module, a WFD communication module, an IrDA module, a BLE communication module, an NFC communication module, a WiBro communication module, a WiMAX communication module, a SWAP communication module, a WiGig communication module, an RF communication module, or a 60-GHz millimeter wave (mmWave) short-range communication module.

In an embodiment, the probe 20 may transmit device information (e.g., identifier (ID) information) about the probe 20 to the ultrasound imaging apparatus 40 by using a first communication method (e.g., BLE), and be wirelessly paired with the ultrasound imaging apparatus 40. In addition, the probe 20 may transmit ultrasound data and/or an ultrasound video to the paired ultrasound imaging apparatus 40.

The device information about the probe 20 may include various pieces of information associated with a serial number, a model name, or battery status of the probe 20.

The ultrasound imaging apparatus 40 may receive device information (e.g., ID information) about the probe 20 from the probe 20 by using the first communication method (e.g., BLE), and be wirelessly paired with the probe 20. In addition, the ultrasound imaging apparatus 40 may transmit an activation signal to the paired probe 20 and receive ultrasound data and/or an ultrasound video from the probe 20. Here, the activation signal may include a signal for controlling an operation of the probe 20.

In an embodiment, the probe 20 may transmit device information (e.g., ID information) about the probe 20 to the ultrasound imaging apparatus 40 by using the first communication method (e.g., BLE), and be wirelessly paired with the ultrasound imaging apparatus 40. In addition, by using a second communication method (e.g., 60-GHz millimeter wave or Wi-Fi), the probe 20 may transmit ultrasound data and/or an ultrasound video to the ultrasound imaging apparatus 40, which is paired with the probe 20 by using the first communication method.

The ultrasound imaging apparatus 40 may receive device information (e.g., ID information) about the probe 20 from the probe 20 by using the first communication method (e.g., BLE), and be wirelessly paired with the probe 20. In addition, the ultrasound imaging apparatus 40 may transmit an activation signal to the paired probe 20 and receive ultrasound data and/or an ultrasound video from the probe 20 by using the second communication method (e.g., 60-GHz millimeter wave or Wi-Fi).

According to an embodiment, the first communication method used for pairing the probe 20 with the ultrasound imaging apparatus 40 may have a lower frequency band than that of the second communication method used for the probe 20 to transmit ultrasound data and/or an ultrasound video to the ultrasound imaging apparatus 40.

The display 140 of the ultrasound imaging apparatus 40 may display user interfaces (UIs) indicating device information about the probe 20. For example, the display 140 may display UIs indicating identification information about the probe 20, a method of pairing with the probe 20, the data communication status between the probe 20 and the ultrasound imaging apparatus 40, a method of performing data communication with the ultrasound imaging apparatus 40, the battery status of the probe 20, or the like.

In a case in which the probe 20 includes the display 112, the display 112 of the probe 20 may display a UI indicating device information about the probe 20. For example, the display 112 may display UIs indicating identification information about the probe 20, a method of pairing with the probe 20, the data communication status between the probe 20 and the ultrasound imaging apparatus 40, a method of performing data communication with the ultrasound imaging apparatus 40, the battery status of the probe 20, or the like.

The communication module 160 may receive a control signal or data from an external device. The processor 120 may control an operation of the ultrasound imaging apparatus 40 according to a control signal received through the communication module 160.

In addition, the processor 120 may transmit a control signal to an external device through the communication module 160 to control the external device according to the transmitted control signal. The external device may operate according to a control signal received from the ultrasound imaging apparatus 40 or may process data received from the ultrasound imaging apparatus 40.

The external device may receive or download a program or an application associated with the ultrasound imaging apparatus 40 from the ultrasound imaging apparatus 40, the probe 20, or a server, and install and execute the program or application on the external device. The ultrasound imaging apparatus 40, the probe 20, or the server providing the program or application may include a recording medium storing instructions, commands, installation files, executable files, data, or the like associated with the program or application. The external device may also be sold with the program or application pre-installed.

The memory 150 may store various pieces of data or programs for driving and controlling the ultrasound imaging apparatus 40, input/output ultrasound data, ultrasound videos, and the like.

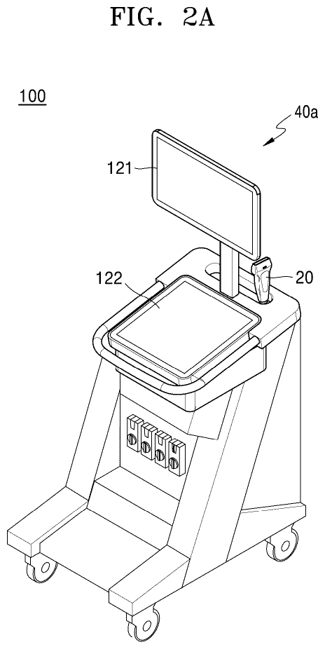

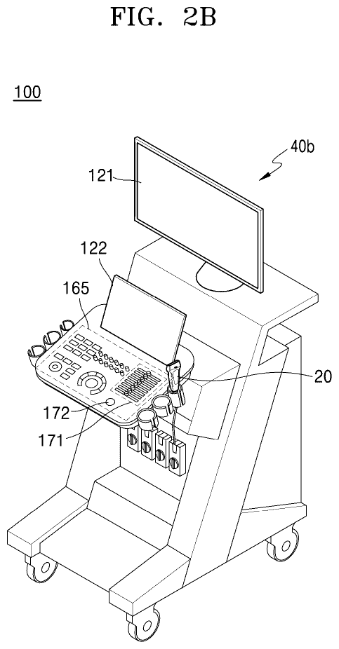

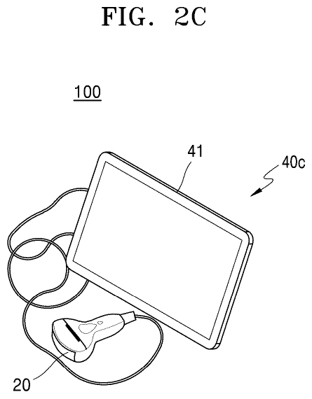

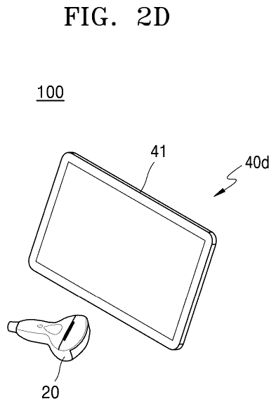

Examples of the ultrasound imaging system 100 according to an embodiment will be described below with reference to FIGS. 2A, 2B, 2C, and 2D.

FIGS. 2A, 2B, 2C, and 2D are diagrams each illustrating an ultrasound imaging apparatus according to an embodiment.

Referring to FIGS. 2A and 2B, ultrasound imaging apparatuses 40a and 40b may each include a main display 121 and a sub-display 122. The main display 121 and the sub-display 122 may correspond to the display 140 of FIGS. 1A and 1B. At least one of the main display 121 or the sub-display 122 may be implemented as a touch screen. At least one of the main display 121 or the sub-display 122 may display an ultrasound video or various pieces of information processed by the ultrasound imaging apparatus 40a or 40b. In addition, at least one of the main display 121 or the sub-display 122 may be implemented as a touch screen, and may provide a graphical user interface (GUI) to receive, from a user, data for controlling the ultrasound imaging apparatus 40a or 40b. For example, the main display 121 may display an ultrasound video, and the sub-display 122 may display a control panel in a GUI form for controlling the display of the ultrasound video. The sub-display 122 may receive data for controlling the display of the video, through the control panel displayed in the GUI form. For example, a time gain compensation (TGC) button, a lateral gain compensation (LGC) button, a Freeze button, a trackball, a jog switch, a knob, or the like may be provided as a GUI on the sub-display 122.

The ultrasound imaging apparatuses 40a and 40b may control the display of the ultrasound video on the main display 121 by using control data that is received as an input. In addition, the ultrasound imaging apparatuses 40a and 40b may be connected to the probe 20 by wire or wirelessly to transmit and receive ultrasound signals to and from an object.

Referring to FIG. 2B, the ultrasound imaging apparatus 40b may further include a control panel 165 in addition to the main display 121 and the sub-display 122. The control panel 165 may include buttons, a trackball, a jog switch, a knob, and the like, and may receive, from the user, data for controlling the ultrasound imaging apparatus 40b. For example, the control panel 165 may include a TGC button 171, a Freeze button 172, and the like. The TGC button 171 is for setting a TGC value for each depth of an ultrasound video. In addition, when an input from the Freeze button 172 is detected while scanning an ultrasound video, the ultrasound imaging apparatus 40b may maintain the display of a frame video at the corresponding time point, capture the frame video at the corresponding time point, or store the frame video at the corresponding time point.

In addition, the buttons, trackball, jog switch, knob, and the like included in the control panel 165 may be provided as GUIs on the main display 121 or the sub-display 122. In addition, the ultrasound imaging apparatuses 40a and 40b may be connected to the probe 20 to transmit and receive ultrasound signals to and from an object.

In addition, the ultrasound imaging apparatuses 40a and 40b may include various types of input/output interfaces such as a speaker, a light-emitting diode (LED), or a vibration device. For example, the ultrasound imaging apparatuses 40a and 40b may output various pieces of information in the form of graphics, sound, or vibration, through the input/output interfaces. In addition, the ultrasound imaging apparatuses 40a and 40b may output various notifications or various pieces of data through the input/output interfaces.

Referring to FIGS. 2C and 2D, ultrasound imaging apparatuses 40c and 40d may be implemented as a portable type. Examples of the portable ultrasound imaging apparatuses 40c and 40d may include a smart phone, a laptop computer, a PDA, or a tablet PC including a probe and an application, but are not limited thereto.

The ultrasound imaging apparatus 40c may include a main body 41. Referring to FIG. 2C, the probe 20 may be connected by wire to one side of the main body 41. To this end, the main body 41 may include a connection terminal to which a cable connected to the probe 20 may be detachably connected. The probe 20 may include a cable including a connection terminal connectable to the main body 41.

Referring to FIG. 2D, the probe 20 may be wirelessly connected to the ultrasound imaging apparatus 40d. The main body 41 may include an input/output interface (e.g., a touch screen). The input/output interface may display an ultrasound video, various pieces of information processed by the ultrasound imaging apparatus 40d, a GUI, or the like.

The ultrasound imaging apparatus 40d and the probe 20 may establish communication or be paired with each other by using short-range wireless communication. For example, the ultrasound imaging apparatus 40d and the probe 20 may perform communication by using Bluetooth, BLE, Wi-Fi, WFD, or the like.

The ultrasound imaging apparatuses 40c and 40d may execute a program or an application associated with the probe 20 to control the probe 20 and to output information associated with the probe 20. The ultrasound imaging apparatuses 40c and 40d may perform operations associated with the probe 20 while communicating with a certain server. The probe 20 may be registered with the ultrasound imaging apparatus 40c or 40d, or with a certain server. The ultrasound imaging apparatuses 40c and 40d may communicate with the registered probe 20 and perform operations associated with the probe 20.

In addition, the ultrasound imaging apparatuses 40c and 40d may include various types of input/output interfaces such as a speaker, an LED, or a vibration device. For example, the ultrasound imaging apparatuses 40c and 40d may output various pieces of information in the form of graphics, sound, or vibration, through the input/output interfaces. In addition, the ultrasound imaging apparatuses 40c and 40d may output various notifications or various pieces of data through the input/output interfaces.

According to an embodiment, the ultrasound imaging apparatus 40a, 40b, 40c, or 40d may process an ultrasound video or obtain additional information from an ultrasound video, by using an artificial intelligence (AI) model. According to an embodiment, the ultrasound imaging apparatus 40a, 40b, 40c, or 40d may generate an ultrasound video or perform processing, such as correction, image quality enhancement, encoding, or decoding, on an ultrasound video, by using an AI model. In addition, according to an embodiment, the ultrasound imaging apparatus 40a, 40b, 40c, or 40d may perform processing, such as defining a baseline, obtaining anatomical information, obtaining lesion information, extracting a surface, defining a boundary, measuring a length, measuring an area, measuring a volume, or generating an annotation, from an ultrasound video by using an AI model.

The AI model may be provided in the ultrasound imaging apparatus 40a, 40b, 40c, or 40d, or a server.

The AI model may be implemented by using various artificial neural network models or deep neural network models. In addition, the AI model may be trained and generated by using various machine learning algorithms or deep learning algorithms. The AI model may be implemented by using models such as a convolutional neural network (CNN), a recurrent neural network (RNN), a generative adversarial network (GAN), or long short-term memory (LSTM).

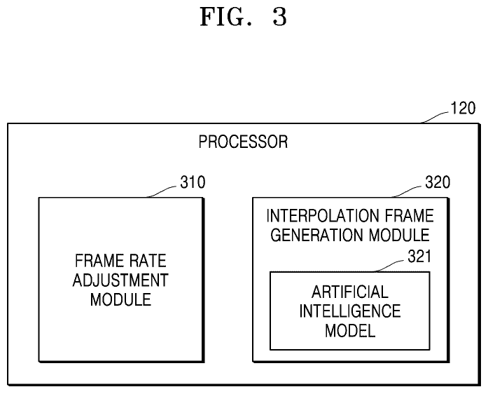

FIG. 3 is a block diagram illustrating the processor 120 of the ultrasound imaging apparatus 40 according to an embodiment.

The ultrasound imaging apparatus 40 may display an ultrasound video. The ultrasound imaging apparatus 40 may obtain ultrasound data through the ultrasound transceiver module 110. The ultrasound data may be data for displaying an ultrasound video. For example, the ultrasound data may be data representing a structure of the object 10 obtained by the ultrasound transceiver module 110.

The ultrasound imaging apparatus 40 may display an ultrasound video through the display 140. The memory 150 of the ultrasound imaging apparatus 40 may store at least one instruction. The processor 120 of the ultrasound imaging apparatus 40 may be electrically connected to the ultrasound transceiver module 110, the display 140, and the memory 150.

The processor 120 may execute the at least one instruction to cause the ultrasound imaging apparatus 40 to obtain ultrasound data. The processor 120 may execute the at least one instruction to cause the ultrasound transceiver module 110 to transmit an ultrasound signal to the object 10. The processor 120 may execute the at least one instruction to obtain an echo signal, which is an ultrasound signal reflected from the object 10 and then received by the ultrasound transceiver module 110.

The processor 120 may execute the at least one instruction to display an ultrasound video through the display 140. The processor 120 may obtain ultrasound data based on the echo signal. The processor 120 may generate an ultrasound video based on the ultrasound data. The processor 120 may display the generated ultrasound video through the display 140.

The processor 120 may execute the at least one instruction to adjust the reproduction speed of a section of interest in an ultrasound video. The section of interest may be a section in the ultrasound video with a degree of importance greater than or equal to a threshold value. For example, the section of interest may be a section in the ultrasound video with a movement greater than or equal to a threshold value. For example, the section of interest may be a section in the ultrasound video where the activity of the object 10 is represented as a value greater than or equal to a threshold value. The processor 120 may adjust the reproduction speed of the section of interest to accurately represent the section of interest. For example, the processor 120 may decrease the reproduction speed of the section of interest to be less than a normal speed.

The processor 120 according to an embodiment may include a frame rate adjustment module 310 and an interpolation frame generation module 320. The processor 120 may process the ultrasound video to enhance the display quality of the ultrasound video. The processor 120 may process the ultrasound video by using the frame rate adjustment module 310 and the interpolation frame generation module 320.

The frame rate adjustment module 310 may adjust the frame rate of an ultrasound video. The frame rate adjustment module 310 may change the frame rate of the ultrasound video for the section of interest from a first frame rate to a second frame rate, based on the adjusted reproduction speed. The second frame rate may be less than the first frame rate. For example, the first frame rate may be 60 Hz, and the second frame rate may be 30 Hz. The frame rate adjustment module 310 may adjust the frame rate of the ultrasound video to adjust the reproduction speed of the ultrasound video.

The processor 120 may execute the at least one instruction to input, to an AI model 321, a plurality of frame images included in the section of interest. The AI model 321 may be included in the interpolation frame generation module 320.

The interpolation frame generation module 320 may generate at least one interpolation frame between the plurality of frame images by using the AI model 321. The at least one interpolation frame may be at least one frame supplementarily inserted between the plurality of frame images. For example, the at least one interpolation frame may be at least one frame that supplements a movement of an object between the plurality of frame images. The at least one interpolation frame may enable more continuous and natural reproduction of the plurality of frame images. The processor 120 may generate at least one interpolation frame between the plurality of frame images by using the AI model 321.

The processor 120 may execute the at least one instruction to generate an interpolated ultrasound video based on the plurality of frame images and the at least one interpolation frame. The processor 120 may generate the interpolated ultrasound video with the at least one interpolation frame inserted between the plurality of frame images.

The processor 120 may execute the at least one instruction to display the interpolated ultrasound video through the display 140. The display 140 may display the interpolated ultrasound video. The ultrasound imaging apparatus 40 according to the disclosure may easily display the object 10 with a complex or fast movement. The ultrasound imaging apparatus 40 according to the disclosure may display a rapidly changing ultrasound video with high quality.

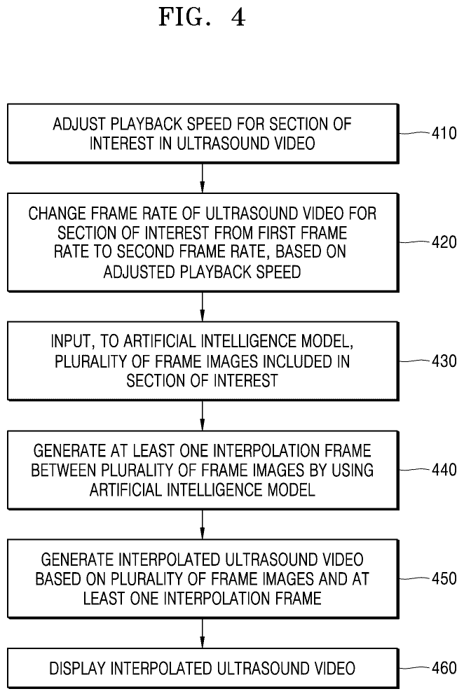

FIG. 4 is a flowchart of a method, performed by the ultrasound imaging apparatus 40, of displaying an ultrasound video, according to an embodiment.

In operation 410, the ultrasound imaging apparatus 40 according to an embodiment may adjust the reproduction speed of a section of interest in an ultrasound video. The processor 120 of the ultrasound imaging apparatus 40 may determine a section of interest based on a movement of an object displayed in the ultrasound video. For example, the processor 120 may determine, as a section of interest, a section in which a movement of the object displayed in the ultrasound video is greater than or equal to a threshold value. The processor 120 may adjust the reproduction speed of the determined section of interest.

In operation 420, the ultrasound imaging apparatus 40 according to an embodiment may change the frame rate of the ultrasound video for the section of interest from a first frame rate to a second frame rate, based on the adjusted reproduction speed. The processor 120 of the ultrasound imaging apparatus 40 may decrease the reproduction speed of the section of interest. For example, the processor 120 of the ultrasound imaging apparatus 40 may decrease the reproduction speed of the section of interest to 0.3 times normal speed, 0.5 times normal speed, or 0.7 times normal speed.

In operation 430, the ultrasound imaging apparatus 40 according to an embodiment may input, to an AI model, a plurality of frame images included in the section of interest.

In operation 440, the ultrasound imaging apparatus 40 according to an embodiment may generate at least one interpolation frame between the plurality of frame images by using the AI model. The processor 120 of the ultrasound imaging apparatus 40 may predict the at least one interpolation frame by using a machine learning model included in the AI model. For example, the processor 120 may predict the at least one interpolation frame suitable to be inserted between the plurality of frame images, by using the machine learning model. The processor 120 may generate the at least one interpolation frame based on whether there is an abnormality in the object displayed in the ultrasound video. For example, when a lesion is found in the object displayed in the ultrasound video, the processor 120 may generate the at least one interpolation frame.

In operation 450, the ultrasound imaging apparatus 40 according to an embodiment may generate an interpolated ultrasound video based on the plurality of frame images and the at least one interpolation frame.

In operation 460, the ultrasound imaging apparatus 40 according to an embodiment may display the interpolated ultrasound video. The ultrasound imaging apparatus 40 according to the disclosure may easily display the object 10 with a complex or fast movement. The ultrasound imaging apparatus 40 according to the disclosure may display a rapidly changing ultrasound video with high quality.

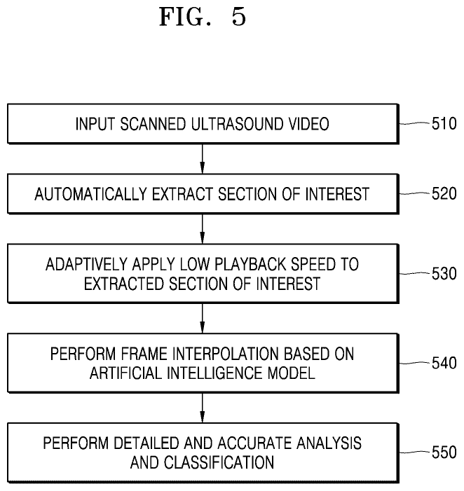

FIG. 5 is a flowchart of a method, performed by the ultrasound imaging apparatus 40, of processing an ultrasound video, according to an embodiment.

In operation 510, the ultrasound imaging apparatus 40 according to an embodiment may input a scanned ultrasound video. The processor 120 of the ultrasound imaging apparatus 40 may cause the ultrasound transceiver module 110 to transmit an ultrasound signal to the object 10. The processor 120 may obtain an echo signal, which is an ultrasound signal reflected from the object 10 and then received by the ultrasound transceiver module 110. The processor 120 may obtain a result of scanning the object 10 based on the echo signal. The processor 120 may generate the ultrasound video based on the obtained scanning result and input the ultrasound video into the memory 150.

In operation 520, the ultrasound imaging apparatus 40 according to an embodiment may automatically extract a section of interest. The processor 120 of the ultrasound imaging apparatus 40 may analyze a degree of movement in the ultrasound video. For example, the processor 120 may analyze a movement of an object included in the ultrasound video. The processor 120 may extract, as a section of interest, a section in which a movement of the object is greater than or equal to a threshold value.

In operation 530, the ultrasound imaging apparatus 40 according to an embodiment may adaptively apply a low reproduction speed to the extracted section of interest. The processor 120 of the ultrasound imaging apparatus 40 may adaptively change the reproduction speed according to the degree of movement in the section of interest. For example, the processor 120 may adaptively adjust the reproduction speed of the section of interest to 0.3 times normal speed, 0.5 times normal speed, or 0.7 times normal speed.

In operation 540, the ultrasound imaging apparatus 40 according to an embodiment may perform frame interpolation based on an AI model. The processor 120 of the ultrasound imaging apparatus 40 may train the AI model by using a plurality of frames included in an ultrasound video. The processor 120 may generate at least one interpolation frame between a plurality of frames by using the trained AI model.

In operation 550, the ultrasound imaging apparatus 40 according to an embodiment may perform detailed and accurate analysis and classification. The processor 120 of the ultrasound imaging apparatus 40 may generate an interpolated frame video based on the plurality of frames and the at least one interpolation frame. The processor 120 may display the interpolated frame video through the display 140. The processor 120 may perform detailed and accurate analysis and classification of a movement of the object by using the interpolated frame video.

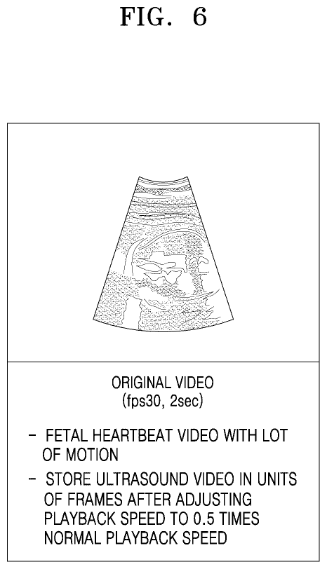

FIG. 6 is a diagram illustrating an original ultrasound video displayed by the ultrasound imaging apparatus 40, according to an embodiment.

The processor 120 of the ultrasound imaging apparatus 40 may display the original ultrasound video through the display 140. The original ultrasound video may be displayed for 2 seconds (2 sec) at 30 frames per second (30 fps). The original ultrasound video may be a video with a lot of motion, indicating a degree of movement within the ultrasound video. For example, the original ultrasound video may be a fetal heartbeat video.

The processor 120 may store an ultrasound video in units of frames. The processor 120 may adjust the reproduction speed of the ultrasound video for detailed review. For example, the processor 120 may store the ultrasound video in units of frames after adjusting the reproduction speed to 0.5 times a normal reproduction speed.

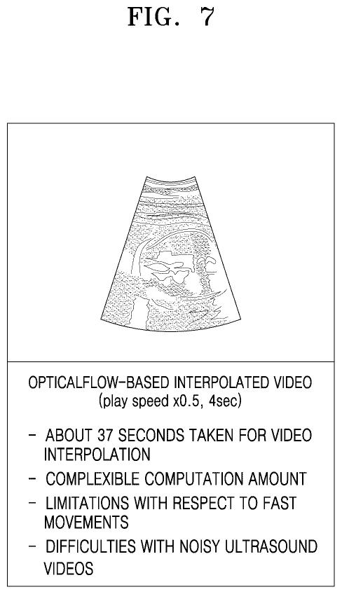

FIG. 7 is a diagram illustrating the ultrasound imaging apparatus 40, according to the disclosure, displaying an ultrasound video that is interpolated according to a comparative example.

The processor 120 of the ultrasound imaging apparatus 40 may display an optical flow-based interpolated ultrasound video according to the comparative example. The processor 120 may reproduce the optical flow-based interpolated ultrasound video for 4 seconds at 0.5 times the normal reproduction speed.

The processor 120 may take about 37 seconds to interpolate the ultrasound video based on an optical flow. The processor 120 may require a complexible computation amount to generate the optical flow-based interpolated ultrasound video. Interpolating an ultrasound video based on an optical flow may exhibit limitations with respect to fast movements. Interpolating an ultrasound video based on an optical flow may present difficulties with noisy ultrasound videos.

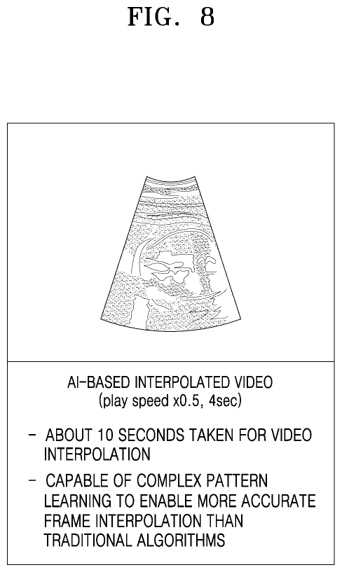

FIG. 8 is a diagram illustrating the ultrasound imaging apparatus 40, according to the disclosure, displaying an ultrasound video that is interpolated according to an embodiment.

The processor 120 of the ultrasound imaging apparatus 40 may display an AI-based interpolated ultrasound video according to an embodiment. The processor 120 may reproduce the AI-based interpolated ultrasound video for 4 seconds at 0.5 times the normal reproduction speed.

The processor 120 may take about 10 seconds to interpolate the ultrasound video based on AI. The processor 120 may be capable of complex pattern learning to generate an AI-based interpolated ultrasound video. When generating an AI-based interpolated ultrasound video, the processor 120 may be capable of more accurate frame interpolation than traditional algorithms.

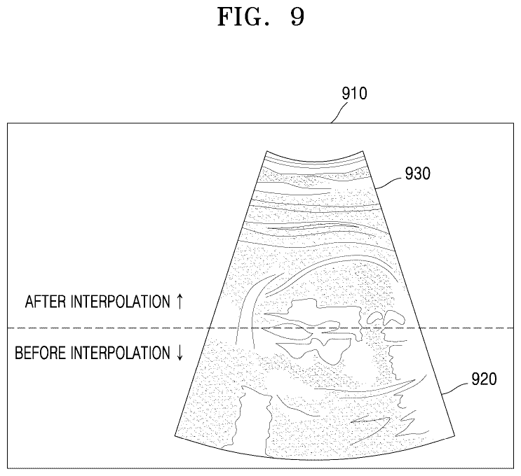

FIG. 9 is a diagram illustrating the ultrasound imaging apparatus 40 interpolating a portion of an ultrasound video 910, according to an embodiment.

The processor 120 of the ultrasound imaging apparatus 40 may interpolate a portion of the ultrasound video 910. The processor 120 may interpolate an upper region of the ultrasound video 910. The processor 120 may reproduce a post-interpolation region 930 with higher quality compared to a pre-interpolation region 920.

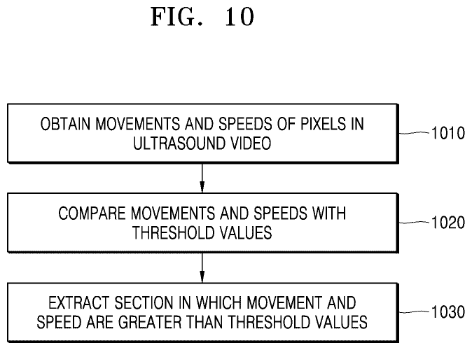

FIG. 10 is a flowchart of a method, performed by the ultrasound imaging apparatus 40, of extracting a section of interest, according to an embodiment.

In operation 1010, the ultrasound imaging apparatus 40 according to an embodiment may compare movements and speeds of pixels in an ultrasound video. The processor 120 of the ultrasound imaging apparatus 40 may apply an algorithm that analyzes movements and speeds of pixels in an ultrasound video. For example, the processor 120 may apply at least one of an optical flow algorithm, a background subtraction algorithm, a frame differencing algorithm, or a phase correlation algorithm, to obtain movements and speeds of pixels in the ultrasound video.

In operation 1020, the ultrasound imaging apparatus 40 according to an embodiment may compare the movements and the speeds with threshold values. The processor 120 of the ultrasound imaging apparatus 40 may define the threshold values for movements and speeds of pixels in the ultrasound video.

In operation 1030, the ultrasound imaging apparatus 40 according to an embodiment may extract a section in which the movement and speed are greater than the threshold values. The processor 120 of the ultrasound imaging apparatus 40 may automatically extract, as a section of interest, a section in which a degree of movement and a speed of pixels are greater than the threshold values. The processor 120 may provide an option to manually set a section of interest for cases in which automatic extraction is difficult or unnecessary.

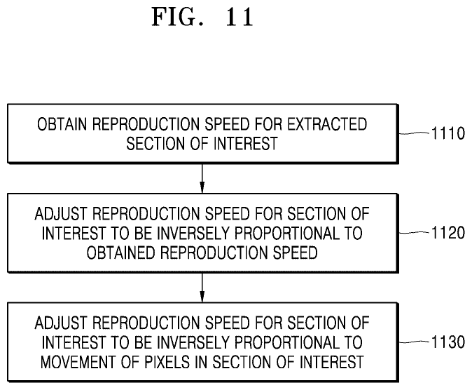

FIG. 11 is a flowchart of a method, performed by the ultrasound imaging apparatus 40, of changing a frame rate, according to an embodiment.

In operation 1110, the ultrasound imaging apparatus 40 according to an embodiment may obtain the reproduction speed of an extracted section of interest. The processor 120 of the ultrasound imaging apparatus 40 may quantify the reproduction speed of the extracted section of interest.

In operation 1120, the ultrasound imaging apparatus 40 according to an embodiment may adjust the reproduction speed of the section of interest to be inversely proportional to the obtained reproduction speed. The processor 120 of the ultrasound imaging apparatus 40 may adjust the reproduction speed of the section of interest to be inversely proportional to the degree of speed of the quantified reproduction speed.

In operation 1130, the ultrasound imaging apparatus 40 according to an embodiment may adjust the reproduction speed of the section of interest to be inversely proportional to a movement of pixels in the section of interest. The processor 120 of the ultrasound imaging apparatus 40 may adjust the reproduction speed of the section of interest to be inversely proportional to the degree of speed of the movement of the pixels. For example, the processor 120 may set the reproduction speed to 1 times normal speed (i.e., the normal speed) when the speed of the movement of the pixels is 10, and may set the reproduction speed to 0.5 times normal speed when the speed of the movement of the pixels is 20. Accordingly, the processor 120 may decrease the reproduction speed of the section of interest that shows a portion in the ultrasound video where a movement of a scanned object is large, such as a heart or blood flow.

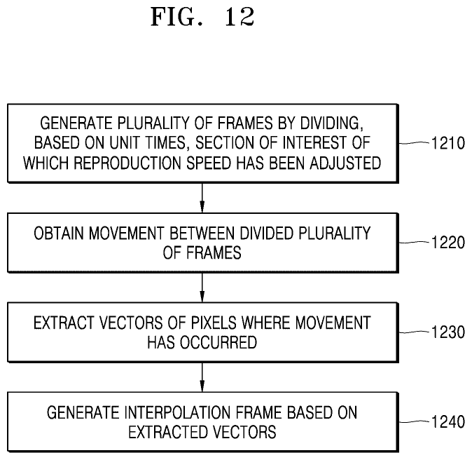

FIG. 12 is a flowchart of a method, performed by the ultrasound imaging apparatus 40, of generating an interpolation frame, according to an embodiment.

In operation 1210, the ultrasound imaging apparatus 40 according to an embodiment may generate a plurality of frames by dividing, based on unit times, a section of interest of which the reproduction speed has been adjusted. The processor 120 of the ultrasound imaging apparatus 40 may construct a separate dataset, considering the characteristics of ultrasound videos, where movement is irregular and continuity is lacking. The processor 120 may divide, along a time axis, each of the plurality of frames in the section of interest of which the reproduction speed has been reduced.

In operation 1220, the ultrasound imaging apparatus 40 according to an embodiment may obtain a movement between the divided plurality of frames. The processor 120 of the ultrasound imaging apparatus 40 may group the plurality of frames into pairs. The processor 120 may define a movement between paired frames.

In operation 1230, the ultrasound imaging apparatus 40 according to an embodiment may extract vectors of pixels where a movement has occurred. The processor 120 of the ultrasound imaging apparatus 40 may use, as labels, the vectors of the pixels where the movement has occurred. For example, the processor 120 may calculate a movement of pixels that have occurred between a first frame and a second frame.

In operation 1240, the ultrasound imaging apparatus 40 according to an embodiment may generate an interpolation frame based on the extracted vectors. The processor 120 of the ultrasound imaging apparatus 40 may predict an interpolation frame to be inserted between a plurality of frames.

For example, the processor 120 may predict a frame (which may be referred to as a 1.5th frame) between the first frame and the second frame, based on a calculation result.

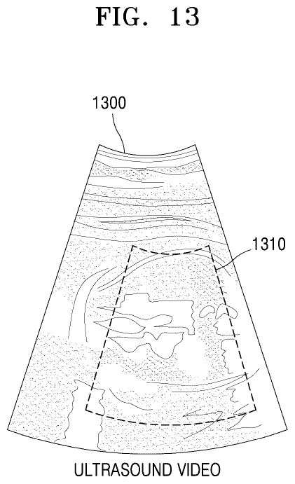

FIG. 13 is a diagram illustrating the ultrasound imaging apparatus 40 setting a region of interest 1310, according to an embodiment.

The ultrasound imaging apparatus 40 according to an embodiment may obtain a motion-mode (M-mode) video based on an ultrasound video 1300 that has been interpolated after decreasing the reproduction speed. The M-mode video may be a video that is a result of obtaining information about a motion in the ultrasound video 1300. For example, the M-mode video may be a video that is a result of obtaining information about a motion by obtaining images of a line included in the ultrasound video 1300 over time. For example, the ultrasound imaging apparatus 40 may obtain an M-mode video based on an interpolated fetal cardiac ultrasound video.

The ultrasound imaging apparatus 40 according to an embodiment may obtain an M-mode video by using at least some of elements of the probe 20. The ultrasound imaging apparatus 40 may obtain an M-mode video by using fewer elements than those used for a B-mode video, which is a general ultrasound video 1300. For example, in a case in which the total number of elements of the probe 20 of the ultrasound imaging apparatus 40 is 192, the ultrasound imaging apparatus 40 may obtain a B-mode video through 192 channels by using all 192 elements of the probe 20. On the contrary, the ultrasound imaging apparatus 40 may obtain an M-mode video by using only some elements of the probe 20. For example, the ultrasound imaging apparatus 40 may obtain an M-mode video for a single scan line by using a single channel. For example, the ultrasound imaging apparatus 40 may obtain an M-mode video by using 5 or fewer channels out of the 192 elements.

The ultrasound imaging apparatus 40 may obtain an M-mode video by setting the region of interest 1310. The region of interest 1310 may be a region to be displayed through an M-mode video among the elements of the probe 20. The ultrasound imaging apparatus 40 may obtain an interpolated M-mode video from ultrasound video data that has been interpolated by setting the region of interest 1310 in the ultrasound video 1300. The ultrasound imaging apparatus 40 may obtain an interpolated M-mode video from interpolated B-mode video data that is previously obtained by setting the region of interest 1310 in the ultrasound video 1300. For example, the ultrasound imaging apparatus 40 may obtain an interpolated M-mode video by setting the region of interest 1310 in an interpolated fetal cardiac ultrasound video.

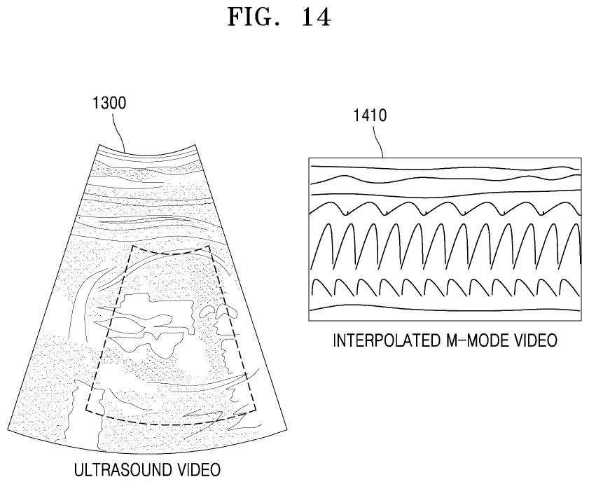

FIG. 14 is a diagram illustrating the ultrasound imaging apparatus 40 displaying an interpolated M-mode video 1410, according to an embodiment.

The ultrasound imaging apparatus 40 may display, on the display 140, the interpolated M-mode video 1410 corresponding to the region of interest 1310. The ultrasound imaging apparatus 40 may display, on the display 140, the interpolated M-mode video 1410 together with the interpolated ultrasound video 1300. The interpolated M-mode video 1410 may represent information about a motion in the interpolated ultrasound video 1300. For example, the interpolated M-mode video 1410 may represent changes in a motion of an object in the interpolated ultrasound video 1300 over time.

The interpolated M-mode video 1410 may represent the changes in the motion of the object in the ultrasound video 1300 with respect to a time axis. The time-axis resolution of the interpolated M-mode video 1410 may be higher than that of the original M-mode video. The interpolated M-mode video 1410 may represent the changes in the motion with respect to the time axis more clearly than the original M-mode video. The interpolated M-mode video 1410 may provide a more accurate diagnosis result to a user of the ultrasound imaging apparatus 40.

The ultrasound imaging apparatus 40 may display a pre-interpolation M-mode video and the interpolated M-mode video 1410 together. The ultrasound imaging apparatus 40 may allow the user of the ultrasound imaging apparatus 40 to easily compare and recognize a disease or an abnormal symptom that is not easy to identify in a pre-interpolation M-mode video. Accordingly, the ultrasound imaging apparatus 40 may provide diagnostic convenience to the user of the ultrasound imaging apparatus 40.

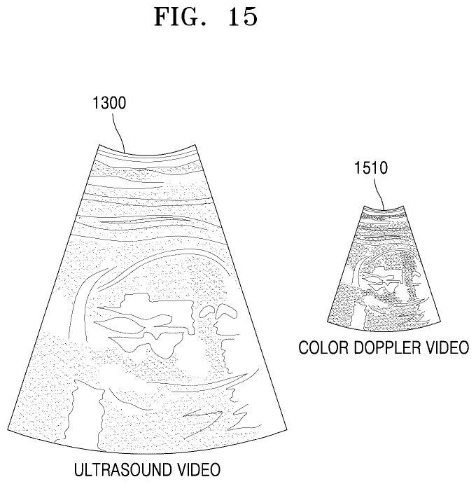

FIG. 15 is a diagram illustrating the ultrasound imaging apparatus 40 displaying a color Doppler video 1510, according to an embodiment.

Ultrasound videos that the ultrasound imaging apparatus 40 displays through the display 140 may include the color Doppler video 1510. The color Doppler video 1510 may be a video in which colors are assigned to distinguish between types and intensities of fluid flows present in an ultrasound video. The ultrasound imaging apparatus 40 may display, through the display 140, the ultrasound video 1300 corresponding to a general B-mode ultrasound video, and the color Doppler video 1510 corresponding to the ultrasound video 1300 together.

The ultrasound imaging apparatus 40 may adjust the reproduction speed of the color Doppler video 1510. The ultrasound imaging apparatus 40 may adjust the reproduction speed of the color Doppler video 1510 by adjusting the reproduction speed of the region of interest 1310 in the ultrasound video 1300.

The ultrasound imaging apparatus 40 may use the AI model 321 to adjust the reproduction speed of the color Doppler video 1510. The ultrasound imaging apparatus 40 may separate a plurality of frame images included in an ultrasound video into B-mode video frames and color Doppler video frames. For example, the ultrasound imaging apparatus 40 may separate a plurality of frame images included in the ultrasound video 1300 into B-mode video frames and color Doppler video frames. The ultrasound imaging apparatus 40 may input, to the AI model 321, each of the B-mode video frames and the color Doppler video frames.

The ultrasound imaging apparatus 40 may generate an interpolated color Doppler video based on ultrasound video frames and color Doppler video frames. For example, the ultrasound imaging apparatus 40 may generate an interpolated color Doppler video by using the AI model 321. The AI model 321 may output an interpolated B-mode video and the interpolated color Doppler video 1510, based on the B-mode video frames and the color Doppler video frames.

The ultrasound imaging apparatus 40 may provide the interpolated color Doppler video 1510 so as to provide the user of the ultrasound imaging apparatus 40 with accurate information associated with a movement in the ultrasound video 1300. For example, the ultrasound imaging apparatus 40 may provide the interpolated color Doppler video 1510 so as to provide the user of the ultrasound imaging apparatus 40 with a more accurate movement of blood flow in the ultrasound video 1300.

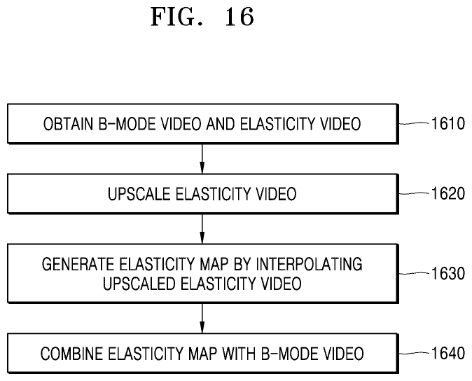

FIG. 16 is a flowchart illustrating the ultrasound imaging apparatus 40 displaying a result of combining an elasticity video with a B-mode video, according to an embodiment. The ultrasound imaging apparatus 40 may fuse two different pieces of video information to simultaneously visualize the anatomical structure and biomechanical properties of an object's tissue. The ultrasound imaging apparatus 40 may combine an elasticity video with a B-mode video to represent the anatomical structure and biomechanical structure of a tissue as a single, integrated ultrasound video.

In operation 1610, the ultrasound imaging apparatus 40 according to an embodiment may obtain a B-mode video and an elasticity video. The B-mode video may be a general ultrasound video. It may be difficult for the B-mode video to directly represent the stiffness or elasticity of an object's tissue. The elasticity video may directly represent the degree of stiffness of the object's tissue. For example, the elasticity video may provide the degree of stiffness of the tissue in the form of a color map or an elasticity map. The elasticity video may visually identify pathological abnormalities of the tissue, such as cancer.

The ultrasound imaging apparatus 40 may perform pre-processing on the obtained B-mode video and elasticity video. For example, the ultrasound imaging apparatus 40 may align the obtained B-mode video and elasticity video. For example, the ultrasound imaging apparatus 40 may remove noise from the obtained B-mode video and elasticity video. For example, the ultrasound imaging apparatus 40 may filter the obtained B-mode video and elasticity video.

In operation 1620, the ultrasound imaging apparatus 40 according to an embodiment may upscale the elasticity video. The resolution of the elasticity video data may be lower than the resolution of the B-mode video data. The ultrasound imaging apparatus 40 may upscale the elasticity video data to a high resolution by using an AI-based interpolation technique. For example, the ultrasound imaging apparatus 40 may upscale, by using a super-resolution model, the resolution of a map representing degrees of stiffness of a lesion included in the elasticity video data, to be equal to the resolution of the B-mode video data.

In operation 1630, the ultrasound imaging apparatus 40 according to an embodiment may generate an elasticity map by interpolating the upscaled elasticity video. The ultrasound imaging apparatus 40 may generate a continuous elasticity map through inter-frame interpolation of the elasticity video. The ultrasound imaging apparatus 40 may improve the temporal resolution of the elasticity map by using an AI-based interpolation technique.

In operation 1640, the ultrasound imaging apparatus 40 according to an embodiment may combine the elasticity map with the B-mode video. The ultrasound imaging apparatus 40 may combine the elasticity video data, which has been upscaled to a high resolution, with the B-mode video. The ultrasound imaging apparatus 40 may generate an interpolated elasticity video based on ultrasound video frames and elasticity video frames. For example, the ultrasound imaging apparatus 40 may generate an interpolated elasticity video by using the AI model 321. The AI model 321 may output an interpolated B-mode video and an interpolated elasticity video, based on B-mode video frames and elasticity video frames.

The ultrasound imaging apparatus 40 may provide the interpolated elasticity video so as to provide the user of the ultrasound imaging apparatus 40 with accurate information associated with a movement and elasticity of an anatomical tissue in the ultrasound video 1300. The ultrasound imaging apparatus 40 may provide accurate clinical information to the user by accurately aligning the interpolated B-mode video and the interpolated elasticity video. For example, the ultrasound imaging apparatus 40 may provide the interpolated elasticity video so as to provide the user of the ultrasound imaging apparatus 40 with a movement of a tissue in the ultrasound video 1300 more accurately.

The ultrasound imaging apparatus 40 may visualize the anatomical structure and biomechanical properties of a tissue in a video obtained by combining the B-mode video with the elasticity video. For example, the ultrasound imaging apparatus 40 may check whether a tissue, in which a lesion of the object included in the ultrasound video 1300 has occurred, matches a tumor boundary line in the ultrasound video 1300. When the tissue in which the lesion has occurred matches the tumor boundary line, the ultrasound imaging apparatus 40 may highlight the matching portion. The ultrasound imaging apparatus 40 may evaluate the clinical significance of a lesion by using AI. The ultrasound imaging apparatus 40 may highlight the clinical significance of the lesion.

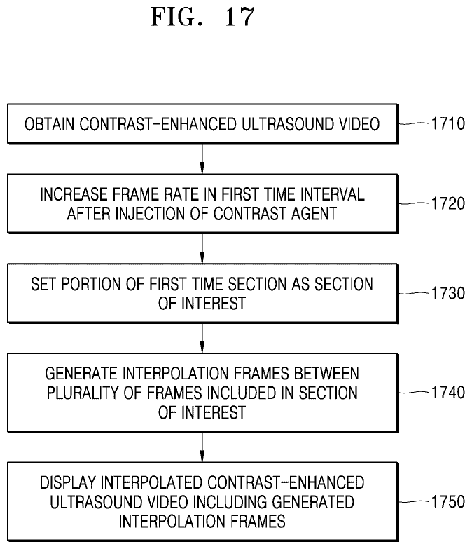

FIG. 17 is a flowchart illustrating the ultrasound imaging apparatus 40 interpolating a contrast-enhanced ultrasound (CEUS) video, according to an embodiment.

In operation 1710, the ultrasound imaging apparatus 40 according to an embodiment may obtain a CEUS video. The CEUS video may be a video in which the visualization of tissue, organs, and blood circulation of an object is improved by using a contrast agent. For example, the contrast agent may be a substance including gas-filled microbubbles. The ultrasound imaging apparatus 40 may obtain a CEUS video in a low-power mode.

In operation 1720, the ultrasound imaging apparatus 40 according to an embodiment may increase the frame rate in a first time section after injection of the contrast agent. The ultrasound imaging apparatus 40 may need to quickly capture changes in blood flow (e.g., wash-in and wash-out) in the CEUS video. The ultrasound imaging apparatus 40 may temporarily increase the frame rate in a particular time section after injecting the contrast agent into an object. For example, the ultrasound imaging apparatus 40 may temporarily increase the frame rate in at least one of an early wash-in section, a peak concentration section, or a wash-out section.

In operation 1730, the ultrasound imaging apparatus 40 according to an embodiment may set a portion of the first time section as a section of interest. The ultrasound imaging apparatus 40 may switch the frame rate of the section of interest, which is set by the user in the CEUS video, to a high frame rate. The ultrasound imaging apparatus 40 may set, as a section of interest, an important section that is automatically detected in the CEUS video, and switch the frame rate of the set section of interest to a high frame rate.

The ultrasound imaging apparatus 40 may pre-process the CEUS video data. Because the ultrasound imaging apparatus 40 obtains a CEUS video in the low-power mode, a signal may be weakened or noise may occur in some situations. The ultrasound imaging apparatus 40 may perform noise reduction processing in a pre-processing operation before interpolation using AI. The ultrasound imaging apparatus 40 may perform separation of tissue signals and contrast signals in the pre-processing operation. The ultrasound imaging apparatus 40 may enhance the quality of an interpolated CEUS video and represent blood flow signals more clearly.

In operation 1740, the ultrasound imaging apparatus 40 according to an embodiment may generate interpolation frames between a plurality of frames included in the section of interest. Even when the ultrasound imaging apparatus 40 captures the section of interest of the CEUS video at a high frame rate, a moment when a movement of microvessels or microbubbles is missed may occur. The ultrasound imaging apparatus 40 may apply an AI model to continuously generate inter-frame interpolation frames for the CEUS video, in order to reduce the occurrence of moments when a movement is missed in the CEUS video.

In operation 1750, the ultrasound imaging apparatus 40 according to an embodiment may display an interpolated CEUS video including the generated interpolation frames. The ultrasound imaging apparatus 40 may generate an interpolated CEUS video based on ultrasound video frames and CEUS video frames. For example, the ultrasound imaging apparatus 40 may generate an interpolated CEUS video by using the AI model 321. The AI model 321 may output an interpolated B-mode video and an interpolated CEUS video, based on B-mode video frames and CEUS video frames.

The ultrasound imaging apparatus 40 may provide the interpolated CEUS video so as to provide the user of the ultrasound imaging apparatus 40 with accurate information associated with a movement in the ultrasound video 1300. For example, the ultrasound imaging apparatus 40 may provide the interpolated CEUS video so as to provide the user of the ultrasound imaging apparatus 40 with a movement of the contrast agent in the ultrasound video 1300 more accurately.

The ultrasound imaging apparatus 40 may generate interpolation frames to visualize wash-in and wash-out processes more smoothly in real time. The ultrasound imaging apparatus 40 may generate interpolation frames to easily represent detailed blood flow changes in the CEUS video. For example, the ultrasound imaging apparatus 40 may generate interpolation frames to easily represent the distribution of microbubbles and the disappearance pattern of microbubbles in the CEUS video.

The disclosure aims to provide a technology for improving the display quality of an ultrasound video through a deep learning-based frame interpolation technique.

According to an embodiment, an ultrasound imaging apparatus for displaying an ultrasound video includes an ultrasound transceiver module configured to obtain volume data for displaying the ultrasound video, a display to display the ultrasound video, a memory storing at least one instruction, and at least one processor electrically connected to the ultrasound transceiver module, the display, and the memory, wherein the at least one processor is configured to execute the at least one instruction to cause the ultrasound imaging apparatus to adjust a reproduction speed of a section of interest in the ultrasound video, change a frame rate of the ultrasound video for the section of interest from a first frame rate to a second frame rate, based on the adjusted reproduction speed, input a plurality of frame images included in the section of interest to an artificial intelligence model, generate at least one interpolation frame between the plurality of frame images by using the artificial intelligence model, generate an interpolated ultrasound video based on the plurality of frame images and the at least one interpolation frame, and display the interpolated ultrasound video through the display.

According to an embodiment, the at least one processor may be further configured to execute the at least one instruction to cause the ultrasound imaging apparatus to decrease the reproduction speed of the section of interest.

According to an embodiment, the at least one processor may be further configured to execute the at least one instruction to cause the ultrasound imaging apparatus to predict the at least one interpolation frame by using a machine learning model included in the artificial intelligence model.

According to an embodiment, the at least one processor may be further configured to execute the at least one instruction to cause the ultrasound imaging apparatus to determine the section of interest based on a movement of an object displayed in the ultrasound video.

According to an embodiment, the at least one processor may be further configured to execute the at least one instruction to cause the ultrasound imaging apparatus to obtain a movement and a speed of pixels in the ultrasound video, compare the movement and the speed with threshold values, and extract a section in which the movement and the speed are greater than the threshold values.

According to an embodiment, the at least one processor may be further configured to execute the at least one instruction to cause the ultrasound imaging apparatus to generate the at least one interpolation frame based on whether there is an abnormality in an object displayed in the ultrasound video.

According to an embodiment, the at least one processor may be further configured to execute the at least one instruction to cause the ultrasound imaging apparatus to adjust the reproduction speed of the section of interest to be inversely proportional to the reproduction speed of the section of interest.

According to an embodiment, the at least one processor may be further configured to execute the at least one instruction to cause the ultrasound imaging apparatus to adjust the reproduction speed of the section of interest to be inversely proportional to a movement of pixels in the section of interest.

According to an embodiment, the at least one processor may be further configured to execute the at least one instruction to cause the ultrasound imaging apparatus to generate a plurality of frames by dividing, based on unit times, the section of interest of which the reproduction speed has been adjusted.

According to an embodiment, the at least one processor may be further configured to execute the at least one instruction to cause the ultrasound imaging apparatus to obtain a movement between the divided plurality of frames, extract vectors of pixels where the movement has occurred, and generate the at least one interpolation frame based on the extracted vectors.

According to an embodiment, the at least one processor may be further configured to execute the at least one instruction to cause the ultrasound imaging apparatus to obtain an interpolated M-mode video from ultrasound video data that is interpolated by setting a region of interest in the ultrasound video.

According to an embodiment, the at least one processor may be further configured to execute the at least one instruction to cause the ultrasound imaging apparatus to separate a plurality of frame images included in the ultrasound video into ultrasound video frames and color Doppler video frames, and generate an interpolated color Doppler video based on the ultrasound video frames and the color Doppler video frames.

According to an embodiment, the at least one processor may be further configured to execute the at least one instruction to cause the ultrasound imaging apparatus to obtain a B-mode video that represents the ultrasound video in a general manner, and an elasticity video, upscale the elasticity video, generate an elasticity map by interpolating the upscaled elasticity video, and combine the elasticity map with the B-mode video.

According to an embodiment, the at least one processor may be further configured to execute the at least one instruction to cause the ultrasound imaging apparatus to obtain a CEUS video, increase the frame rate in a first time section after injection of a contrast agent, set at least a portion of the first time section as the section of interest, generate an interpolation frame between the plurality of frames included in the section of interest, and display an interpolated CEUS video including the generated interpolation frame.

According to an embodiment, a method, performed by an ultrasound imaging apparatus, of displaying an ultrasound video includes adjusting a reproduction speed of a section of interest in the ultrasound video, changing a frame rate of the ultrasound video for the section of interest from a first frame rate to a second frame rate, based on the adjusted reproduction speed, inputting a plurality of frame images included in the section of interest to an artificial intelligence model, generating at least one interpolation frame between the plurality of frame images by using the artificial intelligence model, generating an interpolated ultrasound video based on the plurality of frame images and the at least one interpolation frame, and displaying the interpolated ultrasound video.

According to an embodiment, the changing of the frame rate from the first frame rate to the second frame rate may include decreasing the reproduction speed of the section of interest.

According to an embodiment, the generating of the at least one interpolation frame between the plurality of frame images may include predicting the at least one interpolation frame by using a machine learning model included in the artificial intelligence model.

According to an embodiment, the adjusting of the reproduction speed of the section of interest may include determining the section of interest based on a movement of an object displayed in the ultrasound video.

According to an embodiment, the adjusting of the reproduction speed of the section of interest may include obtaining a movement and a speed of pixels in the ultrasound video, comparing the movement and the speed with threshold values, and extracting a section in which the movement and the speed are greater than the threshold values.

According to an embodiment, the generating of the at least one interpolation frame between the plurality of frame images may include generating the at least one interpolation frame based on whether there is an abnormality in an object displayed in the ultrasound video.

According to an embodiment, the changing of the frame rate from the first frame rate to the second frame rate may include adjusting the reproduction speed of the section of interest to be inversely proportional to the reproduction speed of the section of interest.

According to an embodiment, the changing of the frame rate from the first frame rate to the second frame rate may include adjusting the reproduction speed of the section of interest to be inversely proportional to a movement of pixels in the section of interest.

According to the disclosure, by utilizing an AI model to optimize a frame interpolation process, the accuracy of frame generation may be improved, the computational load for generating interpolation frames may be reduced, and thus, an enhanced-quality ultrasound video may be displayed in real time.

According to the disclosure, the overall quality of an ultrasound video may be improved to enhance the diagnostic capability of the ultrasound imaging apparatus that displays the ultrasound video, and an accurate ultrasound video with enhanced quality may be provided to a user of an ultrasound diagnostic apparatus.

An apparatus, method, or computer program according to an embodiment performs operations associated with AI. The operations associated with AI are performed through a processor and a memory. The processor may perform the operations associated with AI by using one or more processors. In this case, the one or more processors may be general-purpose processors such as a central processing unit (CPU), an application processor (AP), or a digital signal processor (DSP), dedicated graphics processors such as a graphics processing unit (GPU) or a vision processing unit (VPU), or dedicated artificial intelligence processors such as a neural processing unit (NPU). The one or more processors process input data according to a program, instructions, an AI model, or the like stored in the memory.

AI-related program, instructions, or AI model may be generated through machine learning. Here, being generated through learning means that a basic AI model is trained by a learning algorithm using a plurality of pieces of training data, whereby a program, instructions, or an AI model that performs desired characteristics (or purposes) is generated. Such learning may be performed by an apparatus itself where AI operations according to an embodiment are performed, or may be performed through a separate server and/or system. Examples of learning algorithms may include, for example, supervised learning, unsupervised learning, semi-supervised learning, and reinforcement learning, but are not limited thereto.

The AI model may include a plurality of neural network layers. Each of the neural network layers has a plurality of weight values, and performs a neural network arithmetic operation via an arithmetic operation between an arithmetic operation result of a previous layer and the plurality of weight values. The plurality of weight values in each of the plurality of neural network layers may be optimized as a result of training the AI model. The artificial neural network may include, for example, a deep neural network (DNN) and may include, for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), a deep Q-network, or the like, but is not limited thereto.

A machine-readable storage medium may be provided in the form of a non-transitory storage medium. Here, the term ‘non-transitory storage medium’ refers to a tangible device and does not include a signal (e.g., an electromagnetic wave), and the term ‘non-transitory storage medium’ does not distinguish between a case where data is stored in a storage medium semi-permanently and a case where data is stored temporarily. For example, the ‘non-transitory storage medium’ may include a buffer in which data is temporarily stored.

According to an embodiment, the method according to embodiments may be included in a computer program product and provided. The computer program product may be traded as commodities between sellers and buyers. The computer program product may be distributed in the form of a machine-readable storage medium (e.g., a compact disc read-only memory (CD-ROM)), or may be distributed online (e.g., downloaded or uploaded) through an application store or directly between two user devices (e.g., smart phones). In a case of online distribution, at least a portion of the computer program product (e.g., a downloadable app) may be temporarily stored in a machine-readable storage medium such as a manufacturer's server, an application store's server, or a memory of a relay server.

What is claimed is:

1. An ultrasound imaging apparatus for displaying an ultrasound video, the ultrasound imaging apparatus comprising:
an ultrasound transceiver module configured to obtain volume data for displaying the ultrasound video;
a display configured to display the ultrasound video;
a memory storing at least one instruction; and
at least one processor electrically connected to the ultrasound transceiver module, the display, and the memory,
wherein the at least one processor is configured to execute the at least one instruction to cause the ultrasound imaging apparatus to adjust a reproduction speed of a section of interest in the ultrasound video, change a frame rate of the ultrasound video for the section of interest from a first frame rate to a second frame rate, based on the adjusted reproduction speed, input a plurality of frame images included in the section of interest to an artificial intelligence model, generate at least one interpolation frame between the plurality of frame images by using the artificial intelligence model, generate an interpolated ultrasound video based on the plurality of frame images and the at least one interpolation frame, and display the interpolated ultrasound video through the display.

2. The ultrasound imaging apparatus of claim 1, wherein the at least one processor is further configured to execute the at least one instruction to cause the ultrasound imaging apparatus to decrease the reproduction speed of the section of interest.

3. The ultrasound imaging apparatus of claim 1, wherein the at least one processor is further configured to execute the at least one instruction to cause the ultrasound imaging apparatus to predict the at least one interpolation frame by using a machine learning model included in the artificial intelligence model.

4. The ultrasound imaging apparatus of claim 1, wherein the at least one processor is further configured to execute the at least one instruction to cause the ultrasound imaging apparatus to determine the section of interest based on a movement of an object displayed in the ultrasound video.

5. The ultrasound imaging apparatus of claim 1, wherein the at least one processor is further configured to execute the at least one instruction to cause the ultrasound imaging apparatus to obtain a movement and a speed of pixels in the ultrasound video, compare the movement and the speed with threshold values, and extract a section in which the movement and the speed are greater than the threshold values.

6. The ultrasound imaging apparatus of claim 1, wherein the at least one processor is further configured to execute the at least one instruction to cause the ultrasound imaging apparatus to generate the at least one interpolation frame based on whether there is an abnormality in an object displayed in the ultrasound video.

7. The ultrasound imaging apparatus of claim 1, wherein the at least one processor is further configured to execute the at least one instruction to cause the ultrasound imaging apparatus to adjust the reproduction speed of the section of interest to be inversely proportional to the reproduction speed of the section of interest.

8. The ultrasound imaging apparatus of claim 1, wherein the at least one processor is further configured to execute the at least one instruction to cause the ultrasound imaging apparatus to adjust the reproduction speed of the section of interest to be inversely proportional to a movement of pixels in the section of interest.

9. The ultrasound imaging apparatus of claim 1, wherein the at least one processor is further configured to execute the at least one instruction to cause the ultrasound imaging apparatus to generate a plurality of frames by dividing, based on unit times, the section of interest of which the reproduction speed has been adjusted.

10. The ultrasound imaging apparatus of claim 9, wherein the at least one processor is further configured to execute the at least one instruction to cause the ultrasound imaging apparatus to obtain a movement between the generated plurality of frames, extract vectors of pixels where the movement has occurred, and generate the at least one interpolation frame based on the extracted vectors.

11. The ultrasound imaging apparatus of claim 1, wherein the at least one processor is further configured to execute the at least one instruction to cause the ultrasound imaging apparatus to obtain an interpolated M-mode video from ultrasound video data that is interpolated by setting a region of interest in the ultrasound video.

12. The ultrasound imaging apparatus of claim 1, wherein the at least one processor is further configured to execute the at least one instruction to cause the ultrasound imaging apparatus to separate a plurality of frame images included in the ultrasound video into ultrasound video frames and color Doppler video frames, and generate an interpolated color Doppler video based on the ultrasound video frames and the color Doppler video frames.

13. The ultrasound imaging apparatus of claim 1, wherein the at least one processor is further configured to execute the at least one instruction to cause the ultrasound imaging apparatus to obtain a B-mode video that represents the ultrasound video in an original state, and an elasticity video, upscale the elasticity video, generate an elasticity map by interpolating the upscaled elasticity video, and combine the elasticity map with the B-mode video.

14. The ultrasound imaging apparatus of claim 1, wherein the at least one processor is further configured to execute the at least one instruction to cause the ultrasound imaging apparatus to obtain a contrast-enhanced ultrasound (CEUS) video, increase the frame rate in a first time section after injection of a contrast agent, set at least a portion of the first time section as the section of interest, generate an interpolation frame between the plurality of frames included in the section of interest, and display an interpolated CEUS video comprising the generated interpolation frame.

15. A method of displaying an ultrasound video, the method comprising:
adjusting a reproduction speed of a section of interest in the ultrasound video;
changing a frame rate of the ultrasound video for the section of interest from a first frame rate to a second frame rate, based on the adjusted reproduction speed;
inputting a plurality of frame images included in the section of interest to an artificial intelligence model;
generating at least one interpolation frame between the plurality of frame images by using the artificial intelligence model;
generating an interpolated ultrasound video based on the plurality of frame images and the at least one interpolation frame; and
displaying the interpolated ultrasound video.

16. The method of claim 15, wherein the changing of the frame rate from the first frame rate to the second frame rate comprises decreasing the reproduction speed of the section of interest.

17. The method of claim 15, wherein the generating of the at least one interpolation frame between the plurality of frame images comprises predicting the at least one interpolation frame by using a machine learning model included in the artificial intelligence model.

18. The method of claim 15, wherein the adjusting of the reproduction speed of the section of interest comprises determining the section of interest based on a movement of an object displayed in the ultrasound video.

19. The method of claim 15, wherein the adjusting of the reproduction speed of the section of interest comprises:
obtaining a movement and a speed of pixels in the ultrasound video;
comparing the movement and the speed with threshold values; and
extracting a section in which the movement and the speed are greater than the threshold values.

20. The method of claim 15, wherein the generating of the at least one interpolation frame between the plurality of frame images comprises generating the at least one interpolation frame based on whether there is an abnormality in an object displayed in the ultrasound video.
